(19) United States
(12) Patent Application Publication
(10) Pub. No.: US 2026/0198952 A1
(43) Pub. Date: Jul. 16, 2026

(54) Articulating Multifunction Surgical Instruments Such as For Use in Surgical Robotic Systems

(71) Applicant: Covidien LP, Mansfield, MA (US)

(72) Inventors: Thomas E. DROCHNER, Longmont, CO (US); Dylan R. KINGSLEY, Broomfield, CO (US); Christopher T. TSCHUDY, Arvada, CO (US); William R. WHITNEY, Boulder, CO (US); Zachary S. HEILIGER, Nederland, CO (US); Jason G. WEIHE, Longmont, CO (US); Curtis M. SIEBENALLER, Frederick, CO (US)

(21) Appl. No.: 19/135,402

(22) PCT Filed: Nov. 28, 2023
(86) PCT No.: PCT/IB2023/061968

§ 371 Date: Jun. 4, 2025

Related U.S. Application Data
(60) Provisional application No. 63/431,089, filed on Dec. 8, 2022.

(51) Int. Cl. A61B 17/295; A61B 17/00; A61B 34/37

(52) U.S. Cl. CPC A61B 17/295; A61B 34/37; A61B 2017/00336; A61B 2017/00398; A61B 2217/005; A61B 2217/007

(57) ABSTRACT

A surgical instrument includes a housing including four input actuators, a shaft assembly including a proximal shaft and an articulating section disposed at a distal end of the proximal shaft, and an end effector assembly coupled to the articulating section. Articulation of the articulating section articulates the end effector assembly relative to the proximal shaft. The end effector assembly includes a proximal body, first and second jaw members extending distally from the proximal body, and an energizable element selectively deployable relative to the first and second jaw members from a retracted position to a deployed position, wherein the energizable element extends distally from the first and second jaw members. At least one of the first or second jaw members is movable relative to the other and the proximal body from a spaced-apart position to an approximated position to grasp tissue therebetween.

This Application claims priority from U.S. Provisional Patent Application 63/431,089, filed 8 Dec. 2022, the entire content of which is incorporated herein by reference.

### FIELD

This disclosure relates to surgical instruments and systems and, more particularly, to articulating multifunction surgical instruments such as for use in surgical robotic systems.

### BACKGROUND

Robotic surgical systems are increasingly utilized in various different surgical procedures. Some robotic surgical systems include a console supporting a robotic arm. One or more different surgical instruments may be configured for use with the robotic surgical system and selectively mountable to the robotic arm. The robotic arm provides one or more inputs to the mounted surgical instrument to enable operation of the mounted surgical instrument, e.g., to rotate, articulate, and/or actuate the mounted surgical instrument.

As can be appreciated, as additional functional components are added to surgical instruments, for example, articulating surgical instruments such as for use in surgical robotic systems, additional actuation structures, deployable components, and/or electrical connections are required. These additional structures, components, and/or connections may present challenges with respect to spatial constraints and/or mechanical features of the surgical instruments, particularly with respect to any articulating mechanisms of the surgical instruments.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any of the aspects described herein may be used in conjunction with any or all of the other aspects described herein.

Provided in accordance with aspects of this disclosure is a surgical instrument including a housing, a shaft assembly, and an end effector assembly. The housing includes first, second, third, and fourth input actuators. The shaft assembly includes a proximal shaft extending distally from the housing and an articulating section disposed at a distal end of the proximal shaft. The end effector assembly is coupled to the articulating section of the shaft assembly. Articulation of the articulating section of the shaft assembly articulates the end effector assembly relative to the proximal shaft of the shaft assembly. The end effector assembly includes a proximal body, first and second jaw members extending distally from the proximal body, and an energizable element selectively deployable relative to the first and second jaw members from a retracted position to a deployed position, wherein the energizable element extends distally from the first and second jaw members. At least one of the first or second jaw members is movable relative to the other and the proximal body from a spaced-apart position to an approximated position to grasp tissue therebetween.

In an aspect of this disclosure, the surgical instrument further includes an insulative sleeve positioned distally of the articulating section of the shaft assembly and coupled to the energizable element. The insulative sleeve is selectively deployable with the energizable element from the retracted position, wherein the insulative sleeve is disposed about the proximal body, to the deployed position, wherein the insulative sleeve is substantially disposed about the first and second jaw members.

In another aspect of this disclosure, the surgical instrument further includes a knife selectively advanceable between the first and second jaw members to cut tissue grasped therebetween.

In still another aspect of this disclosure, the surgical instrument further includes an energy-based cutting element disposed on one of the first or second jaw members and configured to cut tissue grasped therebetween.

In yet another aspect of this disclosure, the surgical instrument further includes an articulation drive sub-assembly disposed within the housing and operably coupled between the first and second input actuators and the articulating section of the shaft assembly. The articulation drive sub-assembly is configured to articulate the articulating section of the shaft assembly about two perpendicular axes of articulation.

In still yet another aspect of this disclosure, the surgical instrument further includes a jaw drive sub-assembly disposed within the housing and operably coupled between the third input actuator and the first and second jaw members. The jaw drive sub-assembly is configured to move the at least one of the first or second jaw members from the spaced-apart position to the approximated position.

In another aspect of this disclosure, the surgical instrument further includes a deployment sub-assembly disposed within the housing and operably coupled to the jaw drive sub-assembly. An initial actuation of the jaw drive sub-assembly moves the at least one of the first or second jaw members from the spaced-apart position to the approximated position and a further actuation of the jaw drive sub-assembly actuates the deployment sub-assembly to move the energizable element from the retracted position to the deployed position.

In another aspect of this disclosure, the first and second jaw members are maintained substantially stationary during the further actuation of the jaw drive sub-assembly.

In yet another aspect of this disclosure, the deployment sub-assembly includes a slider-crank mechanism.

In still another aspect of this disclosure, the surgical instrument further includes a deployment sub-assembly disposed within the housing and including a motor configured to drive movement of the energizable element from the retracted position to the deployed position.

In another aspect of this disclosure, the housing further includes a plurality of electrical connectors. At least one electrical connector of the plurality of electrical connectors is coupled to the motor to power and control the motor.

In still yet another aspect of this disclosure, the housing is configured to releasably connect to a surgical robotic system. The surgical robotic system is configured to operably couple to and provide rotational inputs to the first, second, third, and fourth input actuators.

In an aspect of this disclosure, the housing does not include any additional input actuators beyond the first, second, third, and fourth input actuators.

Another surgical instrument provided in accordance with this disclosure includes a housing, a shaft assembly, and an end effector assembly. The shaft assembly includes a proximal shaft extending distally from the housing and an articulating section disposed at a distal end of the proximal shaft. First and second fluid lines extend distally through the proximal shaft and articulating section of the shaft assembly. The end effector assembly is coupled to the articulating section of the shaft assembly. Articulation of the articulating section of the shaft assembly articulates the end effector assembly relative to the proximal shaft of the shaft assembly. The end effector assembly includes an insulative member including at least one fluid channel (e.g., first and second fluid channels) fluidly coupled to the first and second fluid lines. The at least one fluid channel (e.g., first fluid channel, in aspects) and first fluid line are adapted to connect to a suction source to suction fluid from a surgical site through the first fluid channel and the at least one fluid channel (e.g., first fluid channel). The at least one fluid channel (e.g., second fluid channel, in aspects) and second fluid line are adapted to connect to a pump to pump fluid through the second fluid line and the at least one fluid channel (e.g., second fluid channel) and into a surgical site.

In an aspect of this disclosure, the end effector assembly further includes an energizable element extending distally from the insulative member. In such aspects, the energizable element may be fixed relative to the insulative member or may extend distally from the insulative member in a deployed position and move between the deployed position and a retracted position, wherein the energizable element is disposed within the insulative member or adjacent a distal end of the insulative member.

In another aspect of this disclosure, the surgical instrument further includes a deployment sub-assembly disposed within the housing and operably coupled to energizable element. The deployment sub-assembly is configured to move the energizable element between the retracted position and the deployed position.

In yet another aspect of this disclosure, the surgical instrument further includes an articulation drive sub-assembly disposed within the housing and operably coupled to the articulating section of the shaft assembly. The articulation drive sub-assembly is configured to articulate the articulating section of the shaft assembly about two perpendicular axes of articulation.

In still another aspect of this disclosure, the housing is configured to releasably connect to a surgical robotic system. In such aspects, the surgical robotic system may be configured to provide an input to the housing to at least one of suction fluid from a surgical site or pump fluid into a surgical site.

The details of one or more aspects of this disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Various aspects and features of this disclosure are described hereinbelow with reference to the drawings wherein like numerals designate identical or corresponding elements in each of the several views.

FIG. 1 is a schematic illustration of a surgical robotic system including a control tower, a console, and one or more surgical robotic arms according to aspects of this disclosure;

FIG. 2 is a perspective view of a surgical robotic arm of the surgical robotic system of FIG. 1 according to aspects of this disclosure;

FIG. 3 is a perspective view of a setup arm with the surgical robotic arm of the surgical robotic system of FIG. 1 according to aspects of this disclosure;

FIG. 4 is a schematic diagram of a computer architecture of the surgical robotic system of FIG. 1 according to aspects of this disclosure;

FIG. 5 is a front, perspective view of a proximal portion of a multifunction surgical instrument provided in accordance with this disclosure and configured for mounting on a robotic arm of a surgical robotic system such as the surgical robotic system of FIG. 1;

FIG. 6 is a rear, perspective view of the proximal portion of the multifunction surgical instrument of FIG. 5;

FIGS. 7A and 7B are perspective views of a distal portion of the multifunction surgical instrument of FIG. 5 disposed in aligned and articulated positions, respectively;

FIG. 8A is a perspective view of the distal portion of the multifunction surgical instrument of FIG. 5 wherein jaw members of the end effector assembly are disposed in a spaced-apart position and a knife is deployed between the jaw members;

FIG. 8B is a perspective view of the distal portion of the multifunction surgical instrument of FIG. 5 wherein the jaw members of the end effector assembly are disposed in an approximated position;

FIG. 8C is a perspective view of the distal portion of the multifunction surgical instrument of FIG. 5 wherein the jaw members of the end effector assembly are disposed in the approximated position and an energizable element is deployed distally beyond the jaw members;

FIG. 9 is a longitudinal, cross-sectional view of the proximal portion of the multifunction surgical instrument of FIG. 5 incorporating a motor assembly for selectively deploying the energizable element;

FIG. 10 is a perspective view of a portion of an actuation assembly of the multifunction surgical instrument of FIG. 5 wherein a jaw drive mechanism of the actuation assembly is operably coupled to a deployment mechanism selectively deploying the energizable element;

FIG. 11 is a perspective view of the end effector assembly of the multifunction surgical instrument of FIG. 5, wherein the jaw members of the end effector assembly are disposed in a spaced-apart position and include an energizable element disposed on one of the jaw members;

FIG. 12 is a perspective view of the end effector assembly of the multifunction surgical instrument of FIG. 5, wherein the jaw members of the end effector assembly are disposed in a spaced-apart position and include an energizable element extending from one of the jaw members;

FIG. 13A is a perspective view of another end effector assembly configured for use with the multifunction surgical instrument of FIG. 5, wherein an energizable element is disposed in a retracted position;

FIG. 13B is a perspective view of the end effector assembly of FIG. 13A, wherein the energizable element is disposed in an extended position; and

FIG. 14 is a perspective view of still another end effector assembly configured for use with the multifunction surgical instrument of FIG. 5.

### DETAILED DESCRIPTION

This disclosure provides articulating multifunction surgical instruments. As described in detail below, the articulating multifunction surgical instruments of this disclosure may be configured for use with a surgical robotic system, which may include, for example, a surgical console, a control tower, and one or more movable carts having a surgical robotic arm coupled to a setup arm. The surgical console receives user inputs through one or more interface devices, which are interpreted by the control tower as movement commands for moving the surgical robotic arm. The surgical robotic arm includes a controller, which is configured to process the movement commands and to generate a torque command for activating one or more actuators of the robotic arm, which, in turn, move the robotic arm in response to the movement commands. Although described hereinbelow in connection with surgical robotic systems, the aspects and features of this disclosure may also be adapted for use with handheld articulating multifunction surgical instruments such as, for example, articulating endoscopic instruments and/or articulating open instruments.

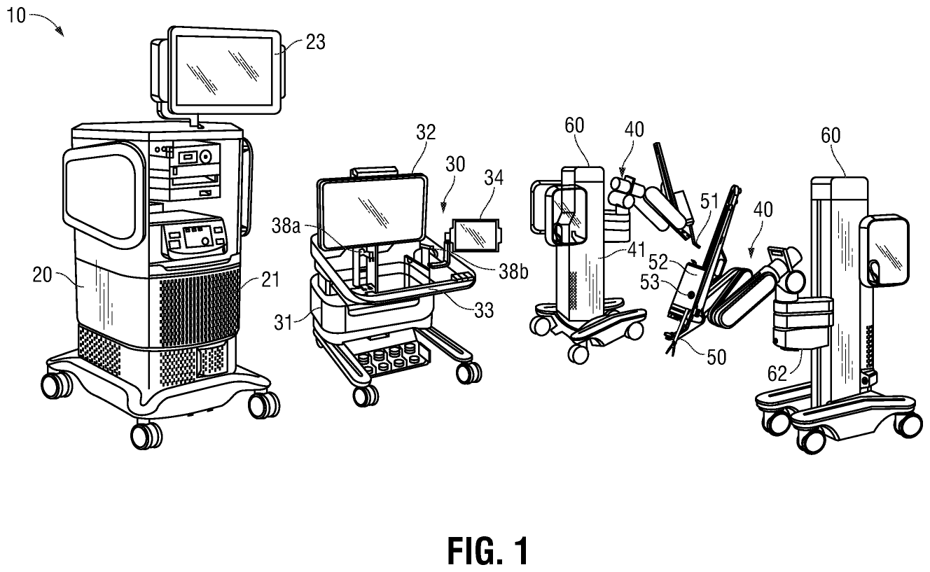

With reference to FIG. 1, a surgical robotic system 10 includes a control tower 20, which is connected to components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The one or more surgical instruments 50 may be configured for use during minimally invasive surgical procedures and/or open surgical procedures. In aspects, one of the surgical instruments 50 may be an endoscope, such as an endoscopic camera 51, configured to provide a video feed for the clinician. In further aspects, one of the surgical instruments 50 may be an energy based surgical instrument such as, for example, an electrosurgical forceps or ultrasonic sealing and dissection instrument configured to seal tissue by grasping tissue between opposing structures and applying electrosurgical energy or ultrasonic energy, respectively, thereto. In yet further aspects, one of the surgical instruments 50 may be a surgical stapler including a pair of jaws configured to clamp tissue, deploy a plurality of tissue fasteners, e.g., staples, through the clamped tissue, and/or to cut the stapled tissue. In still other aspects, one of the surgical instruments 50 may include an energizable element (e.g., a monopolar, bipolar, thermal, microwave, etc. element) configured to treat tissue. Suction and/or irrigation surgical instruments 50 are also contemplated. Other suitable surgical instruments 50 include the multifunction surgical instrument provided in accordance with this disclosure and described in detail hereinbelow.

Endoscopic camera 51, as noted above, may be configured to capture video of the surgical site. In such aspects, the surgical console 30 includes a first display 32, which displays a video feed of the surgical site provided by endoscopic camera 51, and a second display 34, which displays a user interface for controlling the surgical robotic system 10. The first and second displays 32 and 34 may be touchscreen graphical user interface (GUI) displays allowing for receipt of various user inputs.

The surgical console 30 also includes a plurality of user interface devices, such as foot pedals 36 and a pair of handle controllers 38a and 38b which are used by a clinician to remotely control robotic arms 40. The surgical console further includes an armrest 33 used to support clinician's arms while operating the handle controllers 38a and 38b.

The control tower 20 includes a display 23, which may be a touchscreen GUI, and provides outputs to the various GUIs. The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and/or the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by this disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth® (an open wireless protocol for exchanging data over short distances, using short length radio waves, from fixed and mobile devices, creating personal area networks (PANs)), and/or ZigBee® (a specification for a suite of high level communication protocols using small, low-power digital radios based on the IEEE 122.15.4-2003 standard for wireless personal area networks (WPANs)).

The computers 21, 31, 41 may include any suitable processor(s) operably connected to a memory, which may include one or more of volatile, non-volatile, magnetic, optical, quantum, and/or electrical media, such as read-only memory (ROM), random access memory (RAM), electrically-erasable programmable ROM (EEPROM), non-volatile RAM (NVRAM), or flash memory. The processor(s) may be any suitable processor(s) (e.g., control circuit(s)) adapted to perform operations, calculations, and/or set of instructions including, but not limited to, a hardware processor, a field programmable gate array (FPGA), a digital signal processor (DSP), a central processing unit (CPU), a microprocessor, a quantum processor, and combinations thereof. Those skilled in the art will appreciate that the processor may be substituted for by using any logic processor (e.g., control circuit) adapted to execute algorithms, calculations, and/or set of instructions.

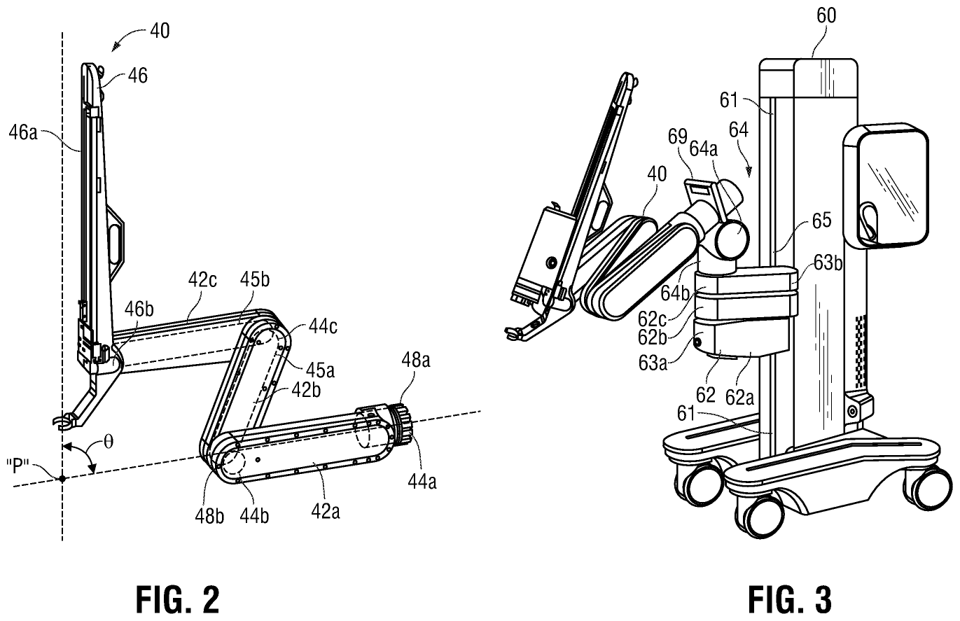

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40. The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62a and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

With reference again to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effectors) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c.

The robotic arm 40 further includes a plurality of manual override buttons 53 disposed on the IDU 52 and the setup arm 62, which may be used in a manual mode. For example, the clinician may press one of the buttons 53 to move the component associated with that button 53.

The joints 44a and 44b include an actuator 48a and 48b configured to drive the joints 44a, 44b, 44c relative to each other through a series of belts 45a and 45b or other mechanical linkages such as drive rods, cables, levers, and/or the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b such that the actuator 48b is configured to rotate each of the links 42b, 42c and the holder 46 relative to one another. More specifically, links 42b, 42c and the holder 46 are passively coupled to the actuator 48b which enforces rotation about a remote center point “P” that lies at an intersection of the first axis defined by the link 42a and the second axis defined by the holder 46. Thus, the actuator 48b controls the angle “θ” between the first and second axes allowing for orientation of the surgical instrument 50. Due to the interlinking of the links 42a, 42b, 42c and the holder 46 via the belts 45a and 45b, the angles between the links 42a, 42b, 42c and the holder 46 are also adjusted in order to achieve the desired angle “θ.” In aspects, some or all of the joints 44a, 44b, 44c may include an actuator to obviate the need for mechanical linkages.

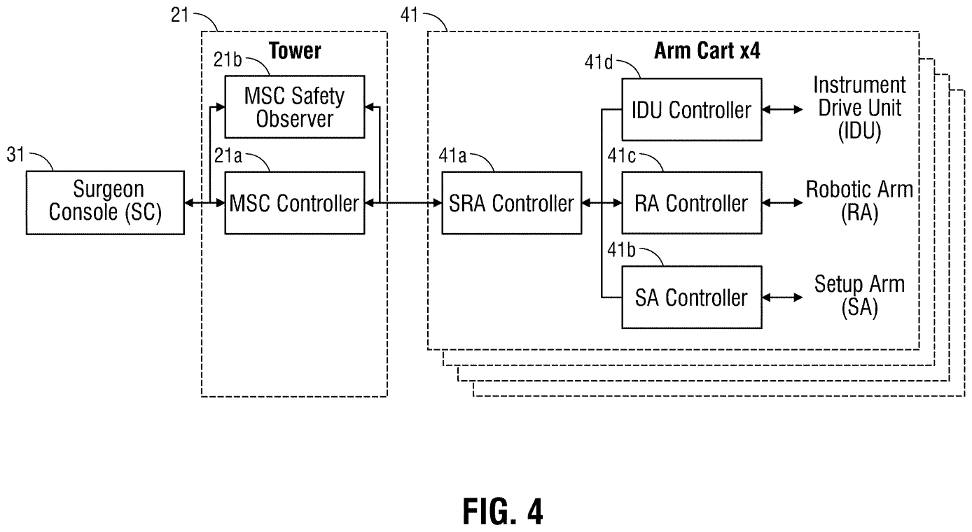

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and/or other inputs. The controller 21a processes these input positions to determine desired drive commands for each joint of the robotic arm 40 and/or the IDU 52 and communicates these to the computer 41 of the robotic arm 40. The controller 21a also receives the actual joint angles and uses this information to determine force feedback commands that are transmitted back to the computer 31 of the surgical console 30 to provide haptic or other feedback through the handle controllers 38a and 38b. The handle controllers 38a and 38b include one or more haptic feedback vibratory devices that output haptic feedback although visual, audible, and/or other feedback is also contemplated. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an IDU controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a communicates the actual joint angles back to the controller 21a.

The setup arm controller 41b controls each of joints 63a and 63b and the rotatable base 64 of the setup arm 62 and calculates desired motor movement commands (e.g., motor torque) for the pitch axis. The setup arm controller 41b also controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

With respect to control of the robotic arm 40, initially, a pose of the handle controller controlling the robotic arm 40, e.g., the handle controller 38a, is transformed into a desired pose of the robotic arm 40 through a hand eye transform function executed by the controller 21a. The hand eye function is embodied in software executable by the controller 21a or any other suitable controller of the surgical robotic system 10. The pose of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limiting mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

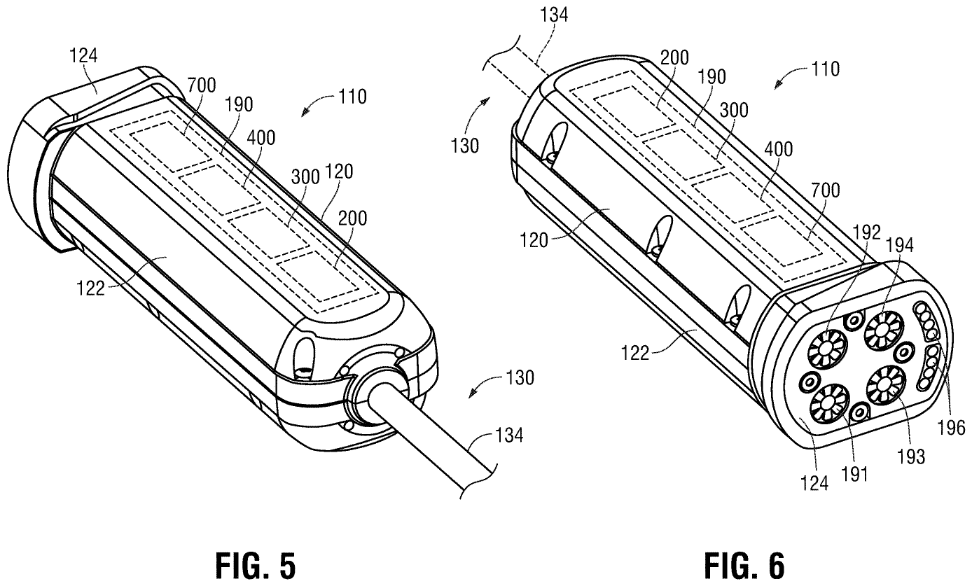

Turning to FIGS. 5-8C, a surgical instrument 110 provided in accordance with this disclosure generally includes a housing 120, a shaft assembly 130 extending distally from housing 120, an end effector assembly 500 extending distally from shaft assembly 130, and an actuation assembly 190 disposed within housing 120 and operably associated with end effector assembly 500. Instrument 110 is detailed herein as an articulating multifunction surgical instrument configured for use with a surgical robotic system, e.g., surgical robotic system 10 (FIG. 1). However, the aspects and features of instrument 110 provided in accordance with this disclosure, as detailed below, are equally applicable for use with other suitable surgical instruments and/or in other suitable surgical systems, e.g., motorized, other power-driven systems, and/or manually actuated surgical systems (including handheld instruments).

Housing 120 of instrument 110 includes a body 122 and a proximal face plate 124 that cooperate to enclose actuation assembly 190 therein. Proximal face plate 124 includes through holes defined therein through which four input actuators or couplers 191-194 of actuation assembly 190 extend. Proximal face plate 124 further mounts a plurality of electrical connectors 196 thereon to enable electrical connection of instrument 110 with a surgical robotic system, e.g., system 10 (FIG. 1), when instrument 110 is mounted on a robotic arm thereof, e.g., to enable communication of data, power, and/or control signals therebetween.

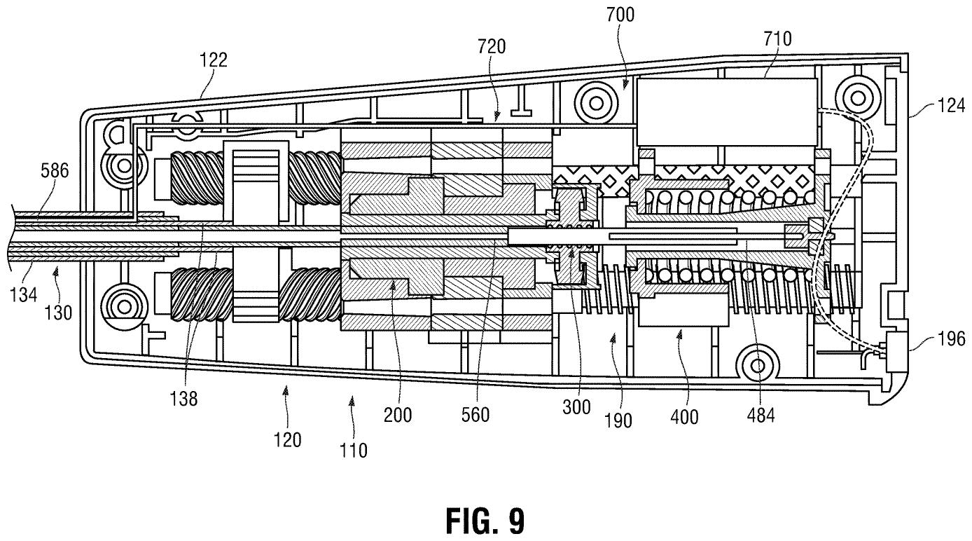

Shaft assembly 130 of instrument 110 includes a proximal shaft 134 and an articulating section 136 disposed between and interconnecting proximal section 134 with end effector assembly 500. Articulating section 136 includes one or more articulating components such as, for example, one or more links, pivots, joints, flexible bodies, etc. A plurality of articulation cables 138 (FIG. 9) or other suitable articulation actuators extend through articulating section 136. More specifically, articulation cables 138 (FIG. 9) may be operably coupled to end effector assembly 500 at the distal ends thereof and extend proximally through articulating section 136 of shaft assembly 130, proximal shaft 134 of shaft assembly 130, and into housing 120, wherein articulation cables 138 (FIG. 9) operably couple with an articulation sub-assembly 200 of actuation assembly 190 to enable selective articulation of end effector assembly 500 relative to proximal shaft 134 and housing 120, e.g., about at least one axis of articulation (yaw articulation, pitch articulation, or both yaw and pitch articulation, for example).

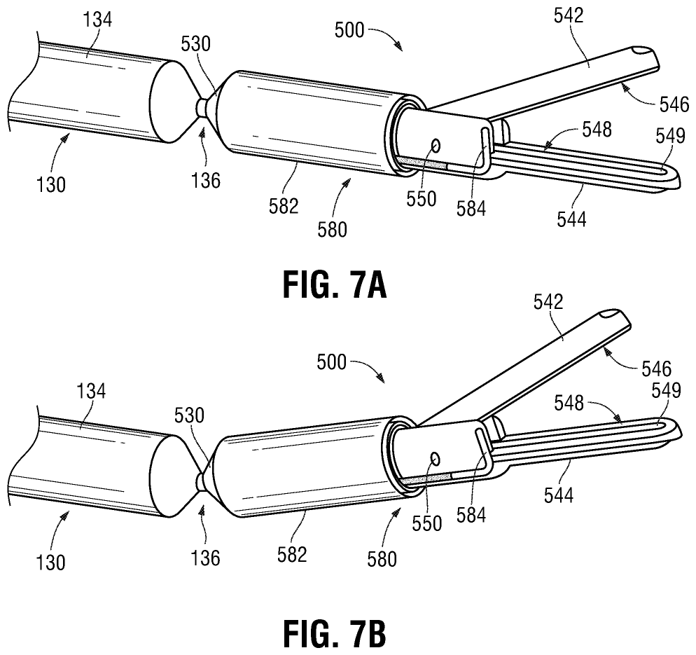

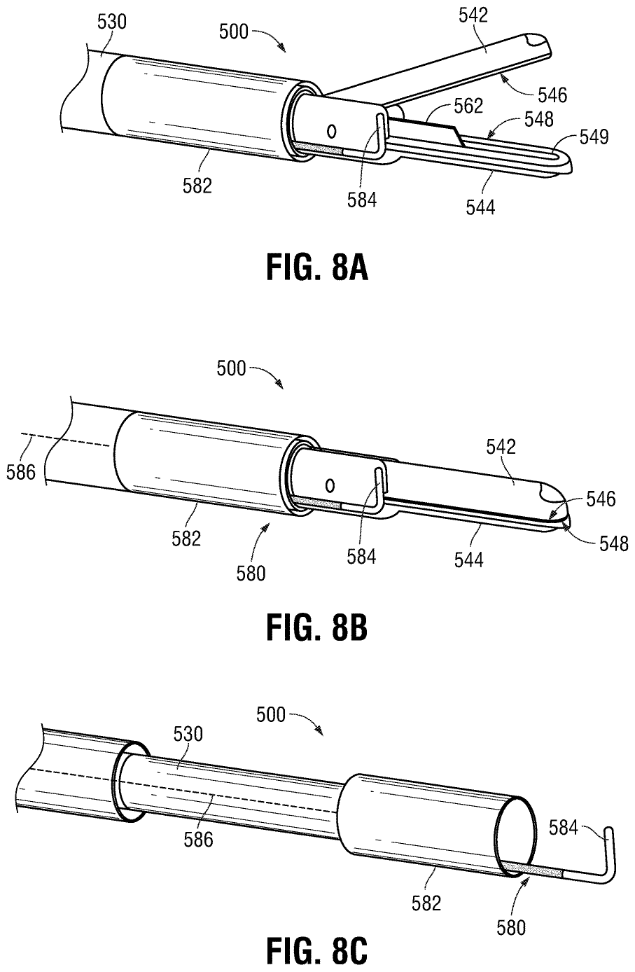

End effector assembly 500 includes a proximal body 530 operably engaged with articulating section 136 of shaft assembly 130. End effector assembly 500 further includes first and second jaw members 542, 544, respectively, pivotably coupled to one another about a pivot 550. Second jaw member 544 is fixed relative to proximal body 530 while first jaw member 542 is pivotable relative to second jaw member 544 and proximal body 530 between a spaced apart position (e.g., an open position of jaw members 542, 544) (FIGS. 7A and 7B) and an approximated position (e.g., a closed position of jaw members 542, 544) (FIG. 8B) for grasping tissue between tissue contacting surfaces 546, 548 of jaw members 542, 544, respectively. As an alternative to this unilateral configuration, a bilateral configuration may be provided whereby both jaw members 542, 544 are pivotable relative to one another and proximal body 530.

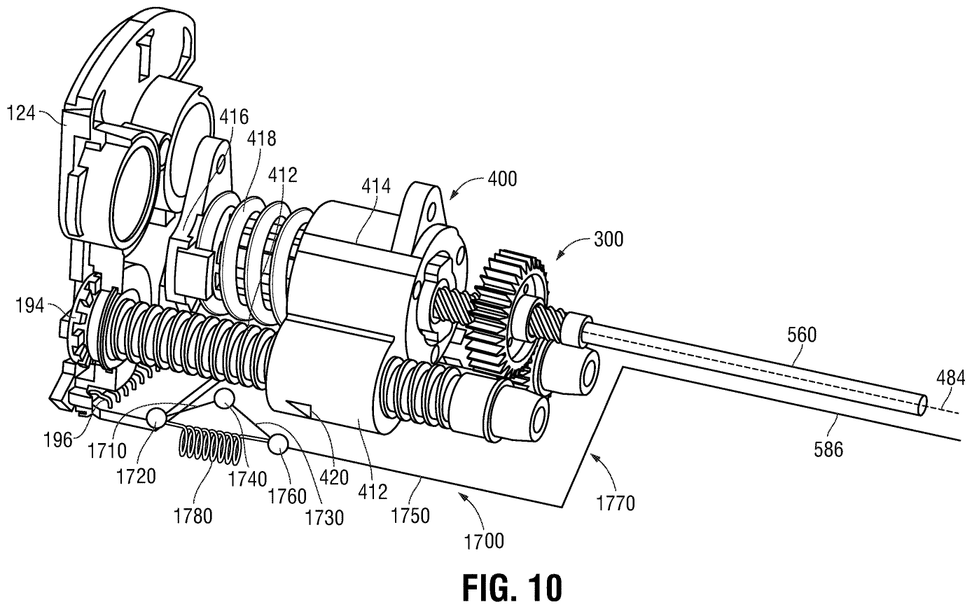

A jaw actuator 484 (FIG. 10) is operably coupled to jaw members 542, 544 (e.g., via a cam-slot mechanism, one or more pulleys, closure-beam, etc.) such that longitudinal translation of jaw actuator 484 (FIG. 10) relative to jaw members 542, 544 pivots jaw member 542 between the spaced-apart and approximated positions (FIGS. 7A and 8B, respectively). More specifically, with momentary reference to FIG. 10, jaw actuator 484 extends proximally from end effector assembly 500 through shaft assembly 130 and into housing 120 wherein jaw actuator 484 is operably coupled with a jaw drive sub-assembly 400 of actuation assembly 190 to enable selective actuation of jaw members 542, 544 between the spaced-apart and approximated positions to grasp tissue therebetween and apply a jaw force within an appropriate jaw force range, as detailed below.

Referring back to FIGS. 5-8C, tissue contacting surfaces 546, 548 of jaw members 542, 544, respectively, are at least partially formed from an electrically conductive material and are energizable to different potentials to enable the conduction of bipolar Radio Frequency (RF) electrical energy through tissue grasped therebetween, although tissue contacting surfaces 546, 548 may alternatively be configured to supply any suitable energy, e.g., thermal, microwave, light, ultrasonic, ultrasound, etc., through tissue grasped therebetween for energy based tissue treatment. Instrument 110 defines a pathway for conductors (not shown) through and/or along housing 120 and shaft 130 to end effector assembly 500 that may include lead wires, contacts, and/or electrically conductive components to enable electrical connection of tissue contacting surfaces 546, 548 of jaw members 542, 544, respectively, to an energy source (not shown), e.g., an electrosurgical generator, for supplying energy to tissue contacting surfaces 546, 548 to treat, e.g., seal, tissue grasped between tissue contacting surfaces 546, 548.

In some configurations, a longitudinally extending knife channel 549 is defined through the tissue contacting surface 546, 548 of one or both jaw members 542, 544. In such aspects, a knife actuator 560 (see FIG. 10) extending from housing 120 through shaft 130 to end effector assembly 500 and a knife 562 disposed within end effector assembly 500 and coupled to knife actuator 560 (FIG. 10) are provided. Knife 562 is selectively translatable between a retracted position, wherein knife 562 is disposed proximally of tissue contacting surfaces 546, 548 of jaw members 542, 544, and an extended position, wherein knife 562 extends through knife channel(s) 549 and between jaw member 542, 544, to cut tissue grasped between tissue contacting surfaces 546, 548 of jaw members 542, 544, respectively. Knife actuator 560 (FIG. 10) is operably coupled to a knife drive sub-assembly 300 of actuation assembly 190 at a proximal end thereof and to knife 562 at a distal end thereof to enable the selective actuation (e.g., translation) of knife actuator 560 (FIG. 10) to, in turn, reciprocate knife 562 between the retracted and extended positions. As an alternative to a longitudinally advanceable knife 562, other suitable mechanical cutters are also contemplated, e.g., guillotine style cutters, rotating cutters, distal-to-proximal motion cutters, etc. Energy-based cutters, e.g., RF electrical cutters, ultrasonic cutters, thermal cutters, light-energy cutters, etc., in static or dynamic configurations, as also contemplated. In such energy-based cutter configurations, instrument 110 defines a suitable conductive pathway (not shown) from an energy source (e.g., generator) through housing 120 and shaft 130 to end effector assembly 500 to enable energization of the energy-base cutter, similarly as detailed above with respect to energizing tissue contacting surfaces 546, 548.

With particular reference to FIGS. 8B and 8C, end effector assembly 500 further includes a deployable assembly 580 including an insulative sleeve 582 and an energizable element 584. Insulative sleeve 582 is slidably disposed about proximal body 530 and is configured for translation about and relative to proximal body 530 between a retracted position (FIG. 8B), where insulative sleeve 582 is disposed proximally of jaw members 542, 544, and a deployed position (FIG. 8C), wherein insulative sleeve 582 is disposed about and substantially surrounds jaw members 542, 544 so as to, in aspects, electrically insulate tissue contacting surfaces 546, 548 of jaw members 542, 544, respectively, from the surroundings of insulative sleeve 582. Energizable element 584 is fixed (e.g., welded, mechanically engaged, or otherwise fixed) relative to insulative sleeve 582 and extends distally therefrom. As a result of the fixed coupling of energizable element 584 and insulative sleeve 582, movement of insulative sleeve 582 between the retracted and deployed positions moves energizable element 584 between a retracted position (FIG. 8B), wherein energizable element 584 is disposed adjacent proximal body 530, and a deployed position (FIG. 8C), wherein energizable element 584 extends distally from jaw members 542, 544.

In aspects, insulative sleeve 582 is fixed about proximal body 530 or omitted and, thus, in such aspects, energizable element 584 is not fixed relative to insulative sleeve 582 but, rather, is movable relative thereto between the retracted and deployed positions. Alternatively or additionally, rather than energizable element 584 moving alongside jaw member 544 from a position proximal thereof to a position distal thereof, energizable element 584 may be deployable from a distal tip of jaw member 544 (or jaw member 542) and/or any other suitable position. Energizable element 584, in other aspects, may be fixed, such as, for example, at or extending from the distal tip of jaw member 544 (or jaw member 542).

Insulative sleeve 582 and/or energizable element 584 are coupled to a deployment actuator 586 at a distal end of deployment actuator 586. Deployment actuator 586 extends proximally from end effector assembly 500 through shaft assembly 130 and housing 120 (FIG. 5) to operably couple to a deployment sub-assembly 700 of actuation assembly 190 at a proximal end of deployment actuator 586 to enable the selective actuation (e.g., translation) of deployment actuator 586 to, in turn, deploy insulative sleeve 582 and energizable element 584 between their respective retracted and deployed positions (FIGS. 8B and 8C, respectively). Energizable element 584 may be energized with any suitable energy, e.g., RF (monopolar or bipolar), ultrasonic, thermal, light-energy, etc., and may define a hook-shaped configuration (as shown) or any other configuration such as, for example, a straight probe, an angled probe, a spatula, an S-curved element, a U-shaped element, combinations thereof, etc. Energizable element 584, regardless of the configuration thereof, is coupled to a suitable conductive pathway (not shown) from an energy source (e.g., generator) through housing 120 and shaft 130 to end effector assembly 500 to enable energization of energizable element 584. In aspects, suitable control algorithms, switching circuits, and/or mechanical connections may be employed to enable energizable element 584 to be energized only when fully deployed, e.g., to the deployed position (FIG. 8C) and/or to enable energization of jaw members 542, 544 only when energizable element 584 is fully retracted, e.g., to the retracted position (FIG. 8B).

Referring generally to FIGS. 5-8C, surgical instrument 110, as detailed above, is a multi-function surgical instrument capable of: energy-based tissue treatment with energizable element 584 (e.g., dissection, scoring, separating tissue layers, spot coagulation, etc.); grasping and manipulating tissue with jaw members 542, 544; energy-based treatment (e.g., sealing) of grasped tissue with tissue contacting surfaces 546, 548 of jaw members 542, 544, respectively; and mechanical cutting of grasped (and, in aspects, previously sealed) tissue with knife 562. Further, each of the actuators, e.g., jaw actuator 484, knife actuator 560, and deployment actuator 586, extends through articulating section 136 of shaft assembly 130 so as to enable movement of jaw members 542, 544 between the spaced-apart and approximated positions, advancement and retraction of knife 562 between jaw members 542, 544, and deployment and retraction of insulative sleeve 582 and energizable element 584 between the retracted and deployed positions regardless of the articulated position of end effector assembly 500 relative to shaft assembly 130.

Actuation assembly 190 is configured to operably interface with a surgical robotic system, e.g., system 10 (FIG. 1), when instrument 110 is mounted on a robotic arm thereof, to enable robotic operation of actuation assembly 190 to provide some or all of the above-detailed functionality. That is, surgical robotic system 10 (FIG. 1) selectively provides inputs, e.g., rotational inputs to input actuators or couplers 191-194 of actuation assembly 190: to actuate articulation sub-assembly 200 to articulate end effector assembly 500 about at least one axis; actuate jaw drive sub-assembly 400 to manipulate jaw members 542, 544; actuate knife drive sub-assembly 300 to advance knife 562 between jaw members 542, 544; and/or actuate deployment sub-assembly 700 to deploy insulative sleeve 582 and energizable element 584.

Sufficient inputs, e.g., an input to each of four input actuators or couplers 191-194, are available where a single input is utilized for actuating each of: articulation sub-assembly 200, jaw drive sub-assembly 400, knife drive sub-assembly 300, and deployment sub-assembly 700. However, with only a single input dedicated, for example, to articulation sub-assembly 200, articulation may be limited to articulation about one axis (pitch or yaw articulation). Thus, where articulation of end effector assembly 500 about at least two axes (e.g., two perpendicular axis such as, for example, enabling pitch and yaw articulation) is provided, thus utilizing two inputs 191, 192 (one for pitch articulation and the other for yaw articulation), an additional or alternative actuation mechanism is required to provide the remainder of the above-detailed functions. In aspects, an additional input is provided (e.g., such that at least five (5) inputs are provided) to enable actuation of deployment sub-assembly 700. In other aspects, such as detailed below, the number of inputs remains unchanged.

Turning to FIGS. 5, 6, and 9, in one configuration in accordance with this disclosure, first and second inputs 191, 192 are coupled to articulation sub-assembly 200, third input 193 is coupled to knife drive sub-assembly 300, and fourth input 194 is coupled to jaw drive sub-assembly 400. Thus, there are no remaining inputs available for deployment sub-assembly 700. As such, in aspects, deployment sub-assembly 700 may include a motor 710 disposed within housing 120. Motor 710 may be a solenoid motor, stepper motor, brushless DC motor, or any other suitable motor. Motor 710 is electrically connected to one or more of electrical connectors 196 to enable electrical connection of motor 710 with the surgical robotic system, e.g., system 10 (FIG. 1), when instrument 110 is mounted on a robotic arm of the surgical robotic system, thus enabling power and control signals to be communicated to motor 710. Motor 710, on an output side thereof, is operably coupled to any suitable connector(s) 720 (e.g., linkages, gears, racks, screws, pivots, sliders, pulleys, etc.) of deployment sub-assembly 700 configured to convert rotational output of motor 710 into translational motion of deployment actuator 586 such that, upon receipt of appropriate electrical signals to drive motor 710, deployment sub-assembly 700 is actuated to deploy or retract insulative sleeve 582 and energizable element 584 (FIGS. 8B and 8C).

With reference to FIGS. 5, 6, and 10, similarly as above, where first and second inputs 191, 192 are coupled to articulation sub-assembly, third input 193 is coupled to knife drive sub-assembly 300, and fourth input 194 is coupled to jaw drive sub-assembly 400, there are no remaining inputs available for deployment sub-assembly 1700. As such, deployment sub-assembly 1700 may be operably coupled to jaw drive sub-assembly 400 such that fourth input 194 enables actuation of both jaw drive sub-assembly 400 and deployment sub-assembly 1700, as detailed below.

Jaw drive sub-assembly 400, in aspects, includes a lead screw 410 operably coupled to fourth input 194 and configured to rotate in response to a rotational input received at fourth input 194, a collar 412 threadingly engaged about lead screw 410 such that rotation of lead screw 410 translates collar 412 along lead screw 410, a first drive body 414 attached to (e.g., formed with, fixed on, or otherwise mechanically engaged with) collar 412 such that translation of collar 412 similarly translates first drive body 414, a second drive body 416 attached to (e.g., formed with, fixed on, or otherwise mechanically engaged with) jaw actuator 484 such that translation of second drive body 416 similarly translates jaw actuator 484, and a spring 418 (e.g., a compression coil spring) disposed between first and second drive bodies 414, 416.

With additional reference to FIGS. 8A and 8B, as a result of the above-detailed configuration of jaw drive sub-assembly 400, a force-limiting feature is realized whereby the force applied to tissue grasped between jaw members 542, 544 is regulated. More specifically, during the initial movement of jaw member 542 towards jaw member 544 from the spaced-apart position towards the approximated position to grasp tissue between tissue contacting surfaces 546, 548, the rotational input received at fourth input 194 rotates lead screw 410 to translate collar 412, thereby translating first drive body 414 towards spring 418 to, in turn, urge spring 418 into second drive body 416 to move second drive body 416, thus translating jaw actuator 484 to pivot jaw member 542 towards jaw member 544. However, when the force applied to tissue grasped between jaw members 542, 544 exceeds a threshold, rather than spring 418 transferring motion to second drive body 416, spring 418 is compressed allowing second drive body 416 to remain stationary (and, thus, the force applied to grasped tissue does not exceed the threshold) despite further rotational input received at fourth input 194 to rotate lead screw 410, translate collar 412, and translate first drive body 414. That is, spring 418 compresses to absorb the translation of first drive body 414 rather than imparting motion to second drive body 416. Accordingly, prior to reaching the jaw force limit, first drive body 414, spring 418, second drive body 416, and jaw actuator 484 move substantially in concert with one another while, after reaching the jaw force limit, second drive body 416 and jaw actuator 484 remain substantially stationary despite further movement of first drive body 414 and the resultant compression of spring 418.

Referring again to FIGS. 5, 6, and 10, jaw drive sub-assembly 400 further includes a wedge 420 or other suitable driver (e.g., a flange, boss, arm, extension, linkage(s), gear(s), etc.) attached to (e.g., formed with, engaged to, or otherwise attached to) collar 412. Wedge 420, as detailed below, operably couples jaw drive sub-assembly 400 with deployment sub-assembly 1700 to enable actuation of deployment sub-assembly 1700 via fourth input 194. Deployment sub-assembly 1700 includes a linkage mechanism, e.g., a four-bar mechanical linkage or slider-crank mechanism, including a first link 1710 pivotably coupled to housing 120 at a first end of first link 1710 via a fixed pivot 1720, a second link 1730 pivotably coupled to a second end of first link 1710 at a first end of second link 1730 via a floating pivot 1740, and a slider 1750 pivotably coupled to a second end of second link 1740 via a sliding pivot 1760. Slider 1760, in turn, is operably coupled to deployment actuator 586 via one or more connectors 1770 (e.g., linkages, gears, racks, screws, pivots, sliders, pulleys, etc.). A spring 1780 biases links 1710, 1730 such that floating pivot 1740 is spaced-apart from a plane defined through fixed pivot 1720 and sliding pivot 1760, corresponding to a proximal position of slider 1750, a proximal position of deployment actuator 586 and, thus, the retracted position of insulative sleeve 582 and energizable element 584 (see FIG. 8B).

In order to deploy insulative sleeve 582 and energizable element 584 (FIG. 8B), a rotational input is provided to fourth input 194, as detailed above, to first move jaw members 542, 544 to the approximated position at the maximum jaw force (see FIG. 8B). Once this position has been achieved, a further rotational input is provided to forth input 194 to further move collar 412 along lead screw 410. As noted above, this motion does not input further force to jaw members 542, 544 (FIG. 8B) due to the compression of spring 418. As collar 412 is moved further along lead screw 410, wedge 420 is urged into contact with second link 1730 and/or floating pivot 1740, thereby urging floating pivot 1740 towards the plane defined through fixed pivot 1720 and sliding pivot 1760 and, as a result, slider 1750 is urged to slide distally. This distal movement of slider 1750, in turn, moves the one or more connectors 1770 to move deployment actuator 586 distally, thereby moving insulative sleeve 582 and energizable element 584 (FIG. 8C) to the deployed position. An opposite rotational input to fourth input 194 displaces wedge 420 from second link 1730 and/or floating pivot 1740, thereby allowing deployment sub-assembly 1700 to return to its initial position under the bias of spring 1780 and, thus, retracting insulative sleeve 582 and energizable element 584 (FIG. 8C) to the retracted position.

It is also contemplated that deployment sub-assembly 1700 be coupled to another sub-assembly to enable actuation of both sub-assemblies with a single input. Further, in aspects, rather than the compression of spring 418 of jaw drive sub-assembly 400 (or the spring of another sub-assembly) enabling or wholly enabling actuation of deployment sub-assembly 1700, jaw drive sub-assembly 400 (or another sub-assembly) may be configured for over-travel beyond the position necessary to, for example, fully close jaw members 542, 544 (with respect to jaw drive sub-assembly 400) or fully retract knife 562 (FIG. 8A) (with respect to knife drive sub-assembly 300). This over-travel thus enables deployment and retraction of deployment sub-assembly 1700 alone or in combination with the compression of a spring (e.g., spring 418).

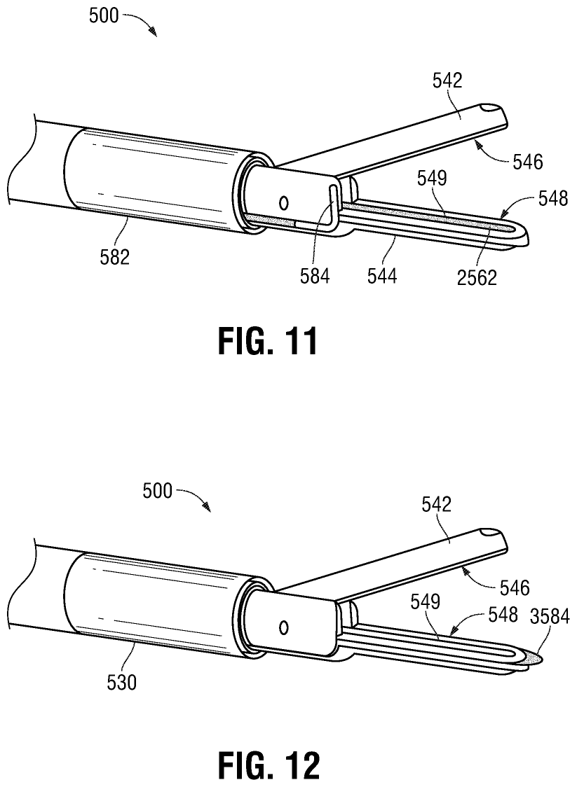

With reference to FIG. 11, in another aspect of this disclosure, in order to dedicate two inputs to articulation, an input to jaw actuation, and an input to deploying and retracting insulative sleeve 582 and energizable element 584 (FIGS. 8B and 8C), while still enabling cutting of grasped tissue, the movable mechanical knife may be replaced with a fixed energy-based cutting element 2562 configured to apply RF (monopolar or bipolar in combination with one or both of tissue contact surfaces 546, 548), ultrasonic, thermal, light, or any other suitable energy to tissue to cut tissue grasped between jaw members 542, 544. Energy-based cutting element 2562 may be disposed within the channel 549 defined within one of the jaw members, e.g., jaw member 544, and is connected, via a suitable conductive pathway (not shown), to an energy source (e.g., generator) to enable energization thereof for cutting tissue. In aspects, energy-based cutting element 2562 may be energized in an open-jaw configuration to enable open tissue dissection and/or other tissue treatment.

Referring to FIG. 12, in another aspect of this disclosure, in order to dedicate two inputs to articulation, an input to jaw actuation, and an input to knife actuation, while still enabling use of an extended energizable element, energizable element 3584 may be fixed relative to and extend distally from jaw member 544. In aspects, energizable element 3584 may replace the insulative sleeve and energizable element and may be utilized with a mechanical knife or an energy-based cutter disposed between jaw members 542, 544. Energizable element 3584 may be utilized and/or configured according to any of the aspects of energizable element 584 (FIGS. 8A-8C) and/or fixed energy-based cutting element 2562 (FIG. 1) as detailed above, or in any other suitable manner.

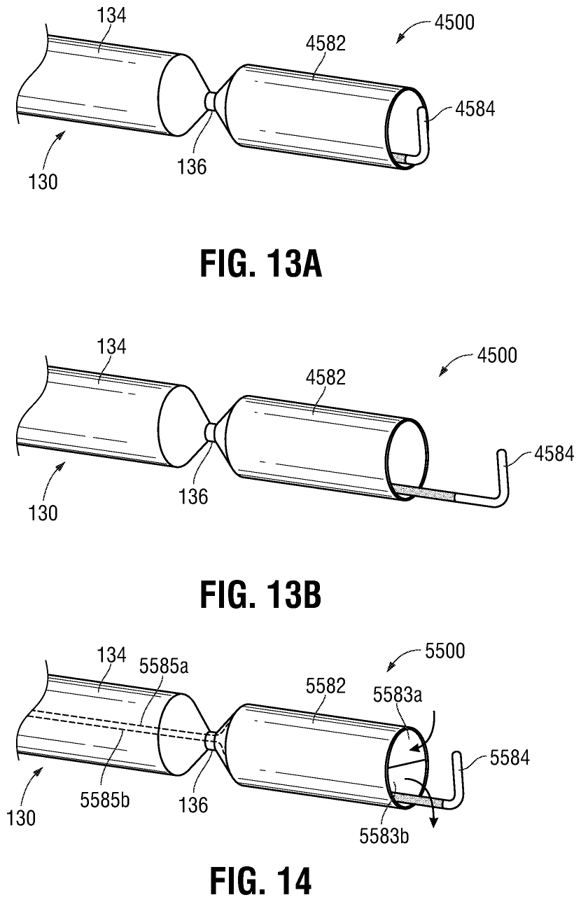

Turning to FIGS. 13A and 13B, in aspects, the jaw members are omitted and end effector assembly 4500 includes insulative member 4582 and energizable element 4584. In such aspects, insulative member 4582 may be an insulative sleeve or an insulative body wherein the interior volume thereof is at least partially filled in or otherwise enclosed. In either configuration, insulative member 4582 may be fixed in position while energizable element 4584 is selectively movable relative to insulative member 4582 between a retracted position (FIG. 13A), wherein energizable element 4584 is disposed within insulative member 4582 or adjacent a distal end thereof, and a deployed position (FIG. 13B), wherein energizable element 4584 is distally-spaced from the distal end of insulative member 4582. As the jaw members (and knife) are omitted in these aspects, there are sufficient inputs to enable articulation of end effector assembly 4500 about multiple axes as well as deployment and retraction of energizable element 4584 with one input left over for an additional function such as, for example, rotation of shaft assembly 130 and/or end effector assembly 4500 about a longitudinal axis thereof.

With reference to FIG. 14, with or without the jaw members, effector assembly 5500 includes insulative sleeve 5582 and energizable element 5584. Insulative sleeve 5582 defines first and second fluid channels 5583a, 5583b, respectively, configured for suction and irrigation, respectively, at a surgical site. More specifically, first and second fluid channels 5583a, 5583b are connected to suction and irrigation lines 5585a, 5585b, respectively, extending proximally from end effector assembly 5500 through shaft assembly 130 and housing 120 (FIG. 5) and operably coupled to a vacuum source (connected to a fluid collection reservoir) and a fluid pump (coupled to a fluid source reservoir), respectively, to enable the suctioning of fluid, tissue, and debris from a surgical site and the pumping of fluid (e.g., saline or other suitable fluid) into the surgical site, respectively. In aspects, one or more pumps (not shown) may be disposed within housing 120 (FIG. 5) and coupled between one or more input actuators or couplers 191-194 (FIG. 6) and suction and/or irrigation lines 5585a, 5585b such that a rotational input provided to the input actuator(s) or coupler(s) 191-194 (FIG. 6) operates the corresponding pump to suction and/or pump fluid along suction and/or irrigation lines 5585a, 5585b, respectively. External pumps are also contemplated. As an alternative to separate first and second fluid channels 5583a, 5583b, a single channel through insulative sleeve 5582 may be utilized for both suction and irrigation, e.g., wherein suction and irrigation lines 5585a, 5585b, respectively, are merged into a single channel.

Energizable element 5584 may be fixed in position relative to insulative sleeve 5582 or movable relative thereto between retracted and deployed positions, similarly as detailed above with respect to energizable element 4584 (FIGS. 13A & 13B). Alternatively or additionally, insulative sleeve 5582 may be movable relative to energizable element 5584 between retracted and deployed positions. Energizable element 5584 may be configured similarly to any of the energizable elements detailed above or in any other suitable manner. In other aspects, energizable element 5584 is omitted.

It will be understood that various modifications may be made to the aspects and features disclosed herein. Therefore, the above description should not be construed as limiting, but merely as exemplifications of various configurations. Those skilled in the art will envision other modifications within the scope and spirit of the claims appended hereto.

What is claimed is:

1. A surgical instrument, comprising:
a housing including first, second, third, and fourth input actuators;
a shaft assembly including a proximal shaft extending distally from the housing and an articulating section disposed at a distal end of the proximal shaft; and
an end effector assembly coupled to the articulating section of the shaft assembly, wherein articulation of the articulating section of the shaft assembly articulates the end effector assembly relative to the proximal shaft of the shaft assembly, the end effector assembly including:
a proximal body;
first and second jaw members extending distally from the proximal body, at least one of the first or second jaw members movable relative to the other and the proximal body from a spaced-apart position to an approximated position to grasp tissue therebetween; and
an energizable element selectively deployable relative to the first and second jaw members from a retracted position to a deployed position, wherein the energizable element extends distally from the first and second jaw members.

2. The surgical instrument according to claim 1, further comprising an insulative sleeve positioned distally of the articulating section of the shaft assembly and coupled to the energizable element, the insulative sleeve selectively deployable with the energizable element from a sleeve retracted position, wherein the insulative sleeve is disposed about the proximal body, to a sleeve deployed position, wherein the insulative sleeve is substantially disposed about the first and second jaw members.

3. The surgical instrument according to claim 1, further comprising a knife selectively advanceable between the first and second jaw members to cut tissue grasped therebetween.

4. The surgical instrument according to claim 1, further comprising an energy-based cutting element disposed on one of the first or second jaw members and configured to cut tissue grasped between the first and second jaw members.

5. The surgical instrument according to claim 1, further comprising:
an articulation drive sub-assembly disposed within the housing and operably coupled between the first and second input actuators and the articulating section of the shaft assembly, wherein the articulation drive sub-assembly is configured to articulate the articulating section of the shaft assembly about two perpendicular axes of articulation.

6. The surgical instrument according to claim 1, further comprising:
a jaw drive sub-assembly disposed within the housing and operably coupled between the third input actuator and the first and second jaw members, wherein the jaw drive sub-assembly is configured to move the at least one of the first or second jaw members from the spaced-apart position to the approximated position.

7. The surgical instrument according to claim 1, further comprising:
a deployment sub-assembly disposed within the housing and operably coupled to the jaw drive sub-assembly, wherein an initial actuation of the jaw drive sub-assembly moves the at least one of the first or second jaw members from the spaced-apart position to the approximated position and wherein a further actuation of the jaw drive sub-assembly actuates the deployment sub-assembly to move the energizable element from the retracted position to the deployed position.

8. The surgical instrument according to claim 7, wherein the first and second jaw members are maintained substantially stationary during the further actuation of the jaw drive sub-assembly.

9. The surgical instrument according to claim 7, wherein the deployment sub-assembly includes a slider-crank mechanism.

10. The surgical instrument according to claim 1, further comprising:
a deployment sub-assembly disposed within the housing, the deployment sub-assembly including a motor configured to drive movement of the energizable element from the retracted position to the deployed position.

11. The surgical instrument according to claim 10, wherein the housing further includes a plurality of electrical connectors, at least one electrical connector of the plurality of electrical connectors coupled to the motor to power and control the motor.

12. The surgical instrument according to claim 1, wherein the housing is configured to releasably connect to a surgical robotic system, the surgical robotic system configured to operably couple to and provide rotational inputs to the first, second, third, and fourth input actuators.

13. The surgical instrument according to claim 1, wherein the housing does not include any additional input actuators beyond the first, second, third, and fourth input actuators.

14. A surgical instrument, comprising:
a housing;
a shaft assembly including a proximal shaft extending distally from the housing and an articulating section disposed at a distal end of the proximal shaft, wherein first and second fluid lines extend distally through the proximal shaft and articulating section of the shaft assembly; and
an end effector assembly coupled to the articulating section of the shaft assembly, wherein articulation of the articulating section of the shaft assembly articulates the end effector assembly relative to the proximal shaft of the shaft assembly, the end effector assembly including:
an insulative member including at least one fluid channel fluidly coupled to the first and second fluid lines, respectively, wherein the at least one fluid channel and the first fluid line are adapted to connect to a suction source to suction fluid from a surgical site through the at least one fluid channel and the first fluid line, and wherein the at least one fluid channel and second fluid line are adapted to connect to a pump to pump fluid through the second fluid line and the at least one fluid channel and into a surgical site; and
an energizable element extending distally from the insulative member.

15. The surgical instrument according to claim 14, wherein the energizable element is fixed relative to the insulative member.

16. The surgical instrument according to claim 14, wherein the energizable element extends distally from the insulative member in a deployed position and is movable between the deployed position and a retracted position, wherein the energizable element is disposed within the insulative member or adjacent a distal end of the insulative member.

17. The surgical instrument according to claim 16, further comprising a deployment sub-assembly disposed within the housing and operably coupled to energizable element, the deployment sub-assembly configured to move the energizable element between the retracted position and the deployed position.

18. The surgical instrument according to claim 14, further comprising:
an articulation drive sub-assembly disposed within the housing and operably coupled to the articulating section of the shaft assembly, wherein the articulation drive sub-assembly is configured to articulate the articulating section of the shaft assembly about two perpendicular axes of articulation.

19. The surgical instrument according to claim 14, wherein the housing is configured to releasably connect to a surgical robotic system.

20. The surgical instrument according to claim 19, wherein the surgical robotic system is configured to provide an input to the housing to at least one of suction fluid from a surgical site or pump fluid into a surgical site.
